(19) United States
(12) Patent Application Publication
(10) Pub. No.: US 2008/0119645 A1
(43) Pub. Date: May 22, 2008

(54) Amidites and Methods of Rna Synthesis

(75) Inventors: Richard H. Griffey, Vista, CA (US); Bruce S. Ross, Princeton, NJ (US); Quanlai Song, Carlsbad, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: ISIS PHARMACEUTICALS, INC., Carlsbad, CA (US)

(21) Appl. No.: 11/568,696

(22) PCT Filed: May 3, 2005
(86) PCT No.: PCT/US05/15240

§ 371 Date: Dec. 17, 2007

Related U.S. Application Data
(60) Provisional application No. 60/568,587, filed on May 5, 2004.

(51) Int. Cl. C07H 21/04; C07H 21/02

(52) U.S. Cl. 536/25.31

(57) ABSTRACT

The present invention is directed to amidites useful in the synthesis of oligonucleotides comprising at least one RN moiety, and to methods of using such amidites in the synthesis of such oligonucleotides. The inventive amidites possess surprising coupling efficiency as compared to prior art amidites, while providing convenient intermediates in the synthesis of oligonucleotides possessing at least one free 2′-OH moiety.

### FIELD OF THE INVENTION

The disclosure herein provides teaching of compounds, compositions and methods of use relating to RNA synthesis.

### BACKGROUND OF THE INVENTION

Oligonucleotides have been used in various biological and biochemical applications. They have been used as primers and probes for the polymerase chain reaction (PCR), as antisense agents used in target validation, drug discovery and development, as ribozymes, as aptamers, and as general stimulators of the immune system. As the popularity of oligonucleotides has increased, the need for producing greater sized batches, and greater numbers of small-sized batches, has increased at pace. Additionally, there has been an increasing emphasis on reducing the costs of oligonucleotide synthesis, and on improving the purity and increasing the yield of oligonucleotide products.

A number of innovations have been introduced to the art of oligonucleotide synthesis. Amongst these innovations have been the development of excellent orthogonal protecting groups, activators, reagents, and synthetic conditions. The oligonucleotides themselves have been subject to a variety of modifications and improvements. Amongst these are chemistries that improve the affinity of an oligonucleotide for a specific target, that improve the stability of an oligonucleotide in vivo, that enhance the pharmacokinetic (PK) and toxicological (Tox) properties of an oligonucleotide, etc. These novel chemistries generally involve a chemical modification to one or more of the constituent parts of the oligonucleotide.

The term “oligonucleotide” thus embraces a class of compounds that include naturally-occurring, as well as modified, oligonucleotides. Both naturally-occurring and modified oligonucleotides have proven useful in a variety of settings, and both may be made by similar processes, with appropriate modifications made to account for the specific modifications adopted. A naturally occurring oligonucleotide, i.e. a short strand of DNA or RNA may be envisioned as being a member of the following generic formulas, denominated oligo-RNA and oligo-DNA, respectively, below:

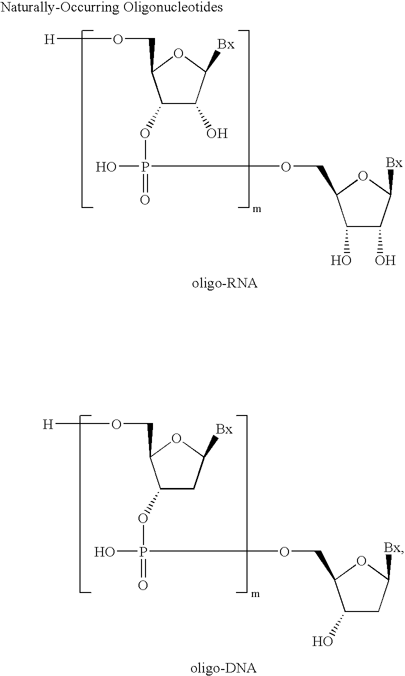

wherein m is an integer of from 1 to about 100, and Bx is one of the naturally occurring nucleobases.

Physiologic pH, an oligonucleotide occurs as the anion, as the phosphate easily dissociates at neutral pH, and an oligonucleotide will generally occur in solid phase, whether amorphous or crystalline, as a salt. Thus, unless otherwise modified, the term “oligonucleotide” encompasses each of the anionic, salt and free acid forms above.

In essence, a naturally occurring oligonucleotide may be thought of as being an oligomer of m monomeric subunits represented by the following nucleotides:

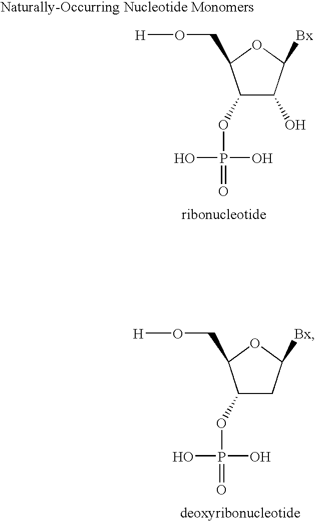

wherein each Bx is a nucleobase, wherein the last residue is a nucleoside (i.e. a nucleotide without the 3′-phosphate group).

As mentioned above, various chemistry modifications have been made to oligonucleotides, in order to improve their affinity, stability, PK, Tox, and other properties. In general, the term oligonucleotide, as now used in the art, encompasses inter alia compounds of the formula:

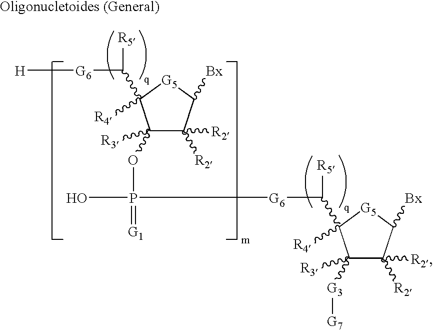

wherein m is an integer from 1 to about 100, each G₁ is O or S, each G₂ is OH or SH, each G₃ is O, S, CH₂, or NH, each G₅ is a divalent moiety such as O, S, CH₂, CFH, CF₂, —CH═CH—, etc., each R₂′ is H, OH, O-rg, wherein rg is a removable protecting group, a 2′-substituent, or together with R₄′ forms a bridge, each R₃′ is H, a substituent, or together with R₄′ forms a bridge, each R₄′ is H, a substituent, together with R₂′ forms a bridge, together with R_{3′} forms a bridge, or together with R₅′ forms a bridge, each q is 0 or 1, each R₅′ is H, a substituent, or together with R₄′ forms a bridge, each G₆ is O, S, CH₂ or NH, and each G₇ is H, PO₃H₂, or a conjugate group, and each Bx is a nucleobase, as described herein (i.e. naturally occurring or modified).

The standard synthetic methods for oligonucleotides include the solid phase methods first described by Caruthers et al. (See, for example, U.S. Pat. No. 5,750,666, incorporated herein by reference, especially columns 3-58, wherein starting materials and general methods of making oligonucleotides, and especially phosphorothioate oligonucleotides, are disclosed, which parts are specifically incorporated herein by reference.) These methods were later improved upon by Köster et al. (See, for example, U.S. Pat. No. RE 34,069, which is incorporated herein by reference, especially columns, wherein are disclosed, which parts are specifically incorporated herein by reference.) These methods have further been improved upon by various inventors, as discussed in more detail below. Methods of synthesizing RNA are disclosed in, inter alia, U.S. Pat. Nos. 6,111,086, 6,008,400, and 5,889,136, each of which is incorporated herein in its entirety. Especially relevant are columns 7-20 of U.S. Pat. No. 6,008,400, which are expressly incorporated herein by reference.

The general process for manufacture of an oligonucleotide by the Köster et al. method may be described as follows:

First, a synthesis support is prepared by covalently linking a suitable nucleoside to a solid support medium (SS) through a linker. Such a synthesis support is as follows:

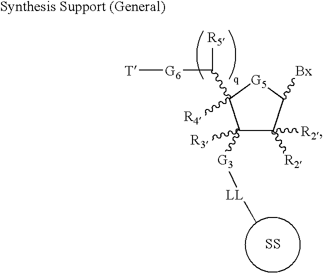

wherein SS is the solid support medium, LL is a linking group that links the nucleoside to the support via G₃. The linking group is generally a di-functional group, covalently binds the ultimate 3′-nucleoside (and thus the nascent oligonucleotide) to the solid support medium during synthesis, but which is cleaved under conditions orthogonal to the conditions under which the 5′-protecting group, and if applicable any 2′-protecting group, are removed. T′ is a removable protecting group, and the remaining variables have already been defined, and are described in more detail herein. Suitable synthesis supports may be acquired from Amersham Biosciences under the brand name Primer Support 200™. The solid support medium having the synthesis support attached thereto may then be swelled in a suitable solvent, e.g. acetonitrile, and introduced into a column of a suitable solid phase synthesis instrument, such as one of the synthesizers available form Amersham Biosciences, such as an ÄKTAoligopilot™, or OligoProcess™ brand DNA/RNA synthesizer.

Synthesis is carried out from 3′- to 5′-end of the oligomer. In each cycle, the following steps are carried out: (1) removal of T′, (2) coupling, (3) oxidation, (4) capping. Each of the steps (1)-(4) may be, and generally is, followed by one or more wash steps, whereby a clean solvent is introduced to the column to wash soluble materials from the column, push reagents and/or activators through the column, or both. The steps (1)-(4) are depicted below:

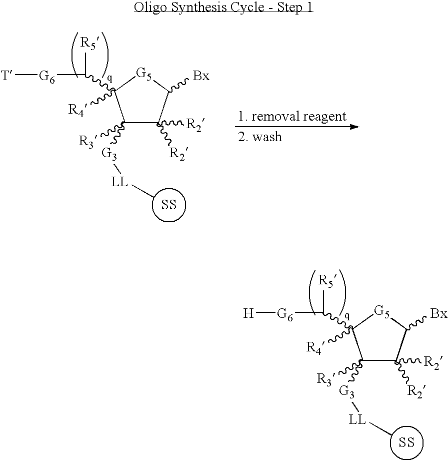

In general, T′ is selected to be removable under conditions orthogonal to those used to cleave the oligonucleotide from the solid support medium at the end of synthesis, as well as those used to remove other protecting groups used during synthesis. An art-recognized protecting group for oligonucleotide synthesis is DMT (4,4′-dimethoxytrityl). The DMT group is especially useful as it is removable under weakly acid conditions. Thus, an acceptable removal reagent is 3% DCA in a suitable solvent, such as acetonitrile. The wash solvent, if used, may conveniently be acetonitrile.

The support may be controlled pore glass or a polymeric bead support. Some polymeric supports are disclosed in the following patents: U.S. Pat. No. 6,016,895; U.S. Pat. No. 6,043,353; U.S. Pat. No. 5,391,667 and U.S. Pat. No. 6,300,486, each of which is specifically incorporated herein by reference.

After removal of protecting group T′, the next step of the synthetic cycle is the coupling of the next nucleoside synthon. This is accomplished by reacting the deprotected support bound nucleoside with a nucleoside phosphoramidite, in the presence of an activator, as shown below:

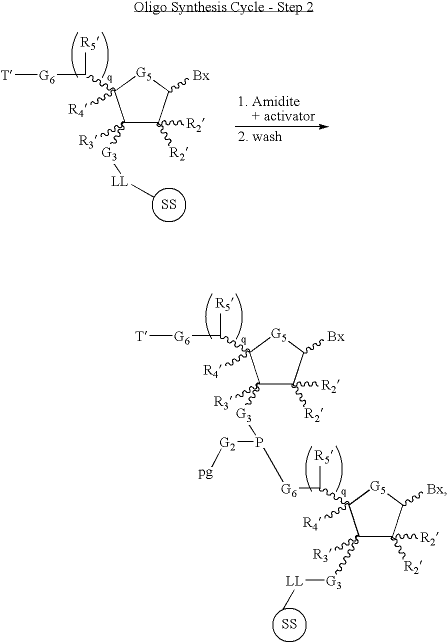

The amidite has the structure:

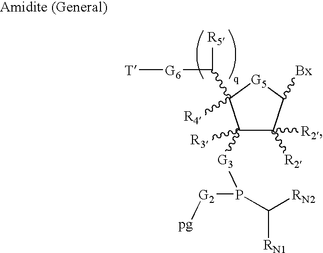

wherein pg is a phosphorus protecting group, such as a cyanoethyl group, and wherein NR_{N1}R_{N2} is an amine leaving group, such as diisopropyl amino, and for teaching of suitable activator (e.g. tetrazole). See, Köster et al., supra, for information on manufacturing of the amidite. Other suitable amidites, and methods of manufacturing amidites, are set forth in the following patents: U.S. Pat. No. 6,133,438; U.S. Pat. No. 5,646,265; U.S. Pat. No. 6,124,450; U.S. Pat. No. 5,847,106; U.S. Pat. No. 6,001,982; U.S. Pat. No. 5,705,621; U.S. Pat. No. 5,955,600; U.S. Pat. No. 6,160,152; U.S. Pat. No. 6,335,439; U.S. Pat. No. 6,274,725; U.S. Pat. No. 6,329,519, each of which is specifically incorporated herein by reference, especially as they relate to manufacture of amidites. Suitable activators are set forth in the Caruther et al. patent and in the Köster et al. patent. Especially suitable activators are set forth in the following patents: U.S. Pat. No. 6,031,092 and U.S. Pat. No. 6,476,216, each of which is expressly incorporated herein by reference.

The next step of the synthesis cycle is oxidation, which indicates that the P(III) species is oxidized to a P(V) oxidation state with a suitable oxidant:

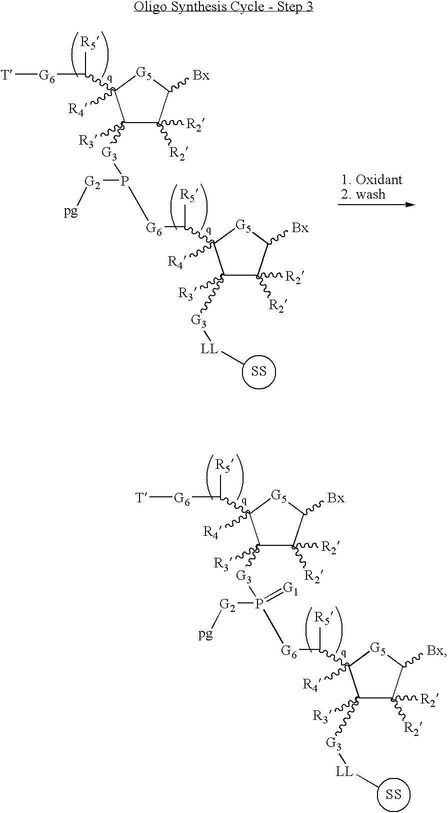

wherein G₁ is O or S.

The oxidant is an oxidizing agent suitable for introducing G₁. In the case where G₁ is oxygen, a suitable oxidant is set forth in the Caruthers et al. patent, above. In cases where G₂ is sulfur, the oxidant may also be referred to as a thiation agent or a sulfur-transfer reagent. Suitable thiation agents include the so-called Beaucage reagent, 3H-1,2-benzothiol, phenylacetyl disulfide (also referred to as PADS; see, for example the patents: U.S. Pat. Nos. 6,114,519 and 6,242,591, each of which is incorporated herein by reference) and thiouram disulfides (e.g. N,N,N′,N′-tetramethylthiouram disulfide, disclosed by U.S. Pat. No. 5,166,387). The wash may be a suitable solvent, such as acetonitrile.

The oxidation step is followed by a capping step, which although not illustrated herein, is an important step for synthesis, as it causes free 5′-OH groups, which did not undergo coupling in step 1, to be blocked from being coupled in subsequent synthetic cycles. Suitable capping reagents are set forth in Caruthers et al., Köster et al., and other patents described herein. Suitable capping reagents include a combination of acetic anhydride and N-methylimidazole.

Synthetic cycle steps (1)-(4) are repeated (if so desired) n−1 times to produce a support-bound oligonucleotide:

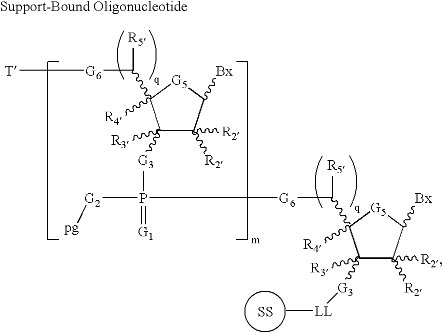

wherein each of the variables is as herein defined.

In general, the protecting group pg may be removed by a method as described by Caruthers et al. or Köster et al., supra. Where pg is a cyanoethyl group, the methodology of Köster et al., e.g. reaction with a basic solution, is generally suitable for removal of the phosphorus protecting group. In some cases it is desirable to avoid formation of adducts such as the N1-cyanoethyl thymidine group. In these cases, it is desirable to include in the reagent a tertiary amine, such as triethylamine (TEA) as taught in U.S. Pat. No. 6,465,628, which is expressly incorporated herein by reference. In general, where the nucleobases are protected, they are deprotected under basic conditions. The deprotected oligonucleotide is cleaved from the support to give the following 5′-protected oligonucleotide:

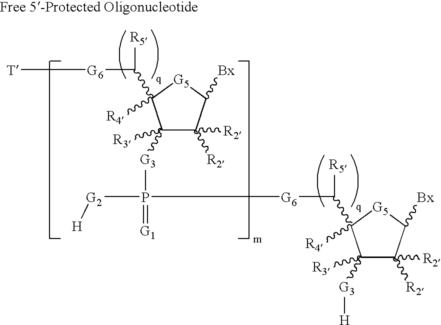

, which may then be purified by reverse phase liquid chromatography, deprotected at the 5′-end in acetic acid, desalted, lyophilized or otherwise dried, and stored in an inert atmosphere until needed. Optionally, the G₃H group may be derivatized with a conjugate group. The resulting oligonucleotide may be visualized as having the formula:

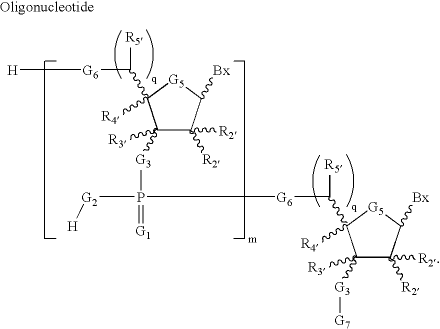

While many improvements have been made in the quality and costs of oligonucleotide synthesis, there still remain a number of improvements to be made.

While many methods and protecting group strategies have been used for the synthesis of RNA, all suffer from drawbacks. These include poor step-wise coupling efficiencies of the amidites, difficulty in removal of the 2′-protecting groups, and lack of compatibility for coupling with other modified nucleoside amidites. For example, the ACE chemistry of Scaringe and co-workers employs a 5′-silyl group, and the 2′-ACE group is acid-labile, conditions not compatible with coupling of 5′-DMT amidites of other nucleosides. See Scaringe, S. A.; Wincott, F. E.; Caruthers, M. H. J. Am. Chem. Soc. 1998, 120, 11820-11821. Other nucleosides with modifications must be prepared with the 5′-silyl protecting group for their incorporation. The 2′-tBDMS protecting group has been used for RNA synthesis for over 25 years. However, it suffers from several deficiencies, including migration of the tBDMS group to the 3′-hydroxyl during preparation of the phosphoramidite, poor step-wise coupling efficiency, and the lability of the terminal 3′-tBDMS group to hydrolysis under acidic or basic conditions. Oligos prepared with 2′-tBDMS groups must undergo multiple chromatography steps following removal of the base protecting groups under basic conditions, removal of the 5′-DMT under acidic conditions, and removal of the 2′-tBDMS using a source of activated fluoride ion.

It can be seen that there exists the need for improved protecting groups which may simultaneously restrict reaction on the protected site but facilitate the reaction at an un-protected site. Moreover, there exists the need for protecting groups which facilitate greater control over reaction order and provide either or both the protection and/or the de-protection of a reaction site with increased control.

These and other benefits are provided according to the present compounds, methods and processes, as described and according to the appended claims.

### SUMMARY OF THE INVENTION

In some embodiments, the present invention provides compounds having the formula:

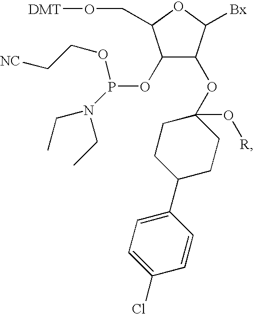

wherein Bx is an optionally protected nucleobase; and R is methyl, ethyl or n-propyl.

In further embodiments, the present invention provides compounds having the formula:

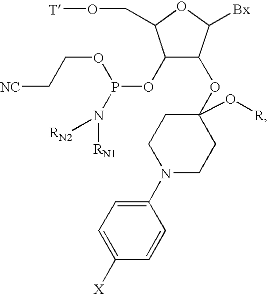

wherein T′ is an acid-labile protecting group; Bx is an optionally protected nucleobase; R is methyl, ethyl, or n-propyl; R_{N1} is H, methyl, ethyl, n-propyl or isopropyl; R_{N2} is, independently of R_{N1} methyl or ethyl; or together R_{N1} and R_{N2} combine to form a pyrrolidinyl, piperidinyl, morpholino or thiomorpholino group; and X is an electron-withdrawing group.

In some embodiments, T′ is 4,4′-dimethoxytriphenylmethyl or pixyl. In some further embodiments, X is F, Cl, Br or CN. In some further embodiments, R is ethyl. In some further embodiments, R_{N1} is methyl, ethyl or isopropyl and R_{N2} is, independently of R_{N1}, methyl or ethyl. In some further embodiments, R_{N1} is methyl and R_{N2} is isopropyl. In some further embodiments, R_{N1} is ethyl and R_{N2} is isopropyl. In some embodiments, R_{N1} and R_{N2} together form a pyrrolidinyl or morpholino moiety.

The present invention also provides processes comprising the steps of:
providing a support-bound species of the formula:

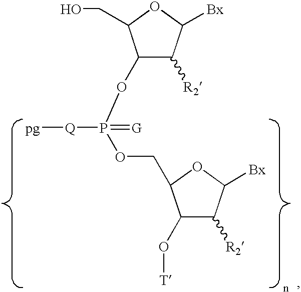

wherein:
n is 0 or a positive integer from 1-100;
each Bx is an optionally protected nucleobase;
each G is O or S;
each Q is O or S;
each pg is H or a protecting group;
each R_{2′} is H, a 2′-deoxy-2′-substituent, or a protected OH group; and
T′ is a support medium or a linker covalently linked to a support medium;
   reacting said support-bound species with an amidite of formula:

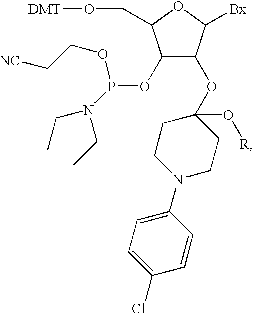

wherein:
Bx is an optionally protected nucleobase;
DMT is the 4,4′-dimethoxytrityl group; and
R is methyl, ethyl or n-propyl;
   to form a support-bound phosphityl compound of formula:

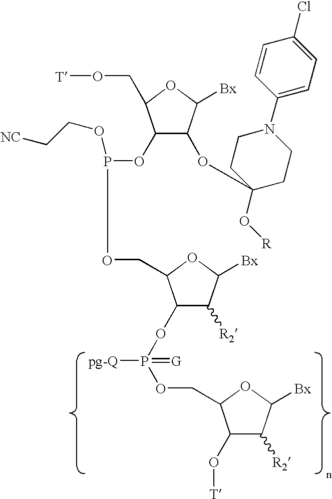

and
(c) oxidizing or sulfurizing the support-bound phosphityl compound to form a phosphotriester compound of formula:

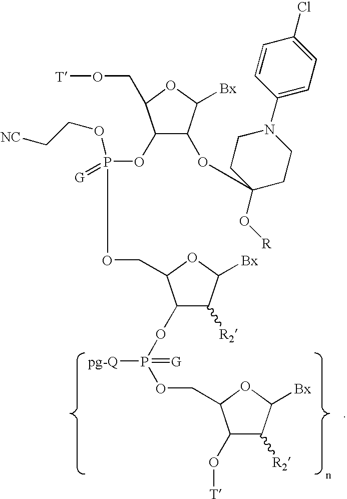

In some embodiments, R is ethyl. In some further embodiments, each Q is O, and each pg is cyanoethyl. In some further embodiments, the process further comprising repeating steps (a)-(c) a plurality of times. In still further embodiments, the process further comprises cleaving the phosphotriester compound from the support medium. In still further embodiments, the process further comprises the step of (d) capping unreacted support bound hydroxyl groups.

In some further embodiments, the present invention provides processes comprising:
(a) providing a support-bound species of the formula:

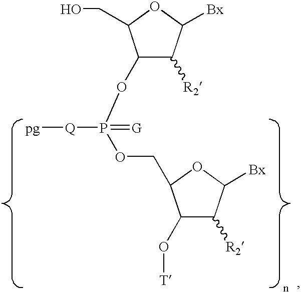

wherein:
   n is 0 or a positive integer from 1 to 100;
   each Bx is an optionally protected nucleobase;
   each G is O or S;
   each Q is O or S;
   each pg is H or a protecting group;
   each R_{2′} is H, a 2′-deoxy-2′-substituent, or a protected OH group; and
   T′ is a support medium or a linker covalently linked to a support medium;
(b) reacting said support-bound species with an amidite of formula:

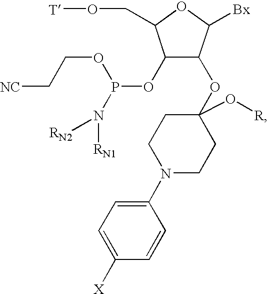

wherein:
   T′ is an acid-labile protecting group;
   Bx is an optionally protected nucleobase;
   R is methyl, ethyl, or n-propyl;
   R_{N1} is H, methyl, ethyl, n-propyl or isopropyl;
   R_{N2} is, independently of RN, methyl or ethyl;
      or together R_{N1} and R_{N2} combine to form a pyrrolidinyl, piperidinyl, morpholino or thiomorpholino group; and
   X is an electron-withdrawing group;
to form a support-bound phosphityl compound of formula:

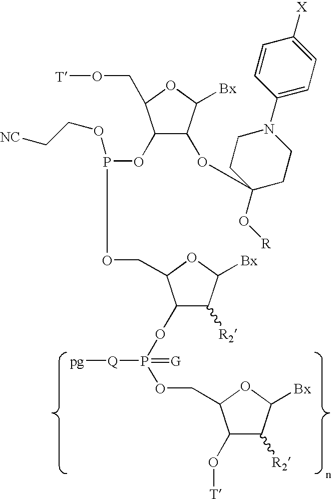

and
oxidizing or sulfurizing the support-bound phosphityl compound to form a phosphotriester compound of formula:

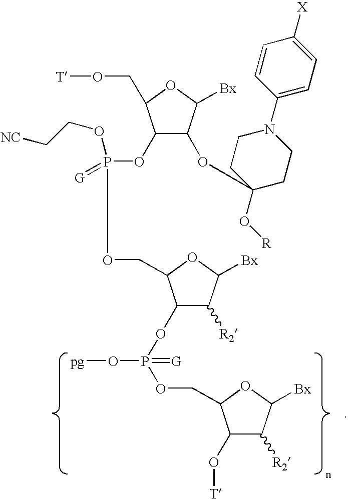

In some embodiments, R is ethyl. In some further embodiments, each Q is O, and each pg is cyanoethyl. In some embodiments, R_{N1} is methyl, ethyl or isopropyl, and R_{N2} is, independently of R_{N1}, methyl or ethyl. In some further embodiments, R_{N1} is methyl and R_{N2} is isopropyl. In some further embodiments, R_{N1} is ethyl and R_{N2} is isopropyl. In some further embodiments, the process further comprises repeating steps (a)-(c) a plurality of times. In still further embodiments, the process further comprises cleaving the phosphotriester compound from the support medium. In still further embodiments, the process further comprises the step of (d) capping unreacted support bound hydroxyl groups.

In some embodiments of the preceding compounds and processes, Bx is U, T or optionally protected G, A, C or 5-methyl C. In further embodiments of the preceding compounds and processes, Bx is optionally protected G. In further embodiments of the preceding compounds and processes, Bx is optionally protected A. In further embodiments of the preceding compounds and processes, Bx is optionally protected C or 5-methyl C. In further embodiments of the preceding compounds and processes, Bx is U or T. In some embodiments wherein Bx is protected G, Bx is G protected with phenylacetyl. In some embodiments wherein Bx is protected A, Bx is A protected with pivolyl. In some embodiments wherein Bx is protected C or protected 5-methyl C, Bx is C or 5-methyl C protected with phenylacetyl.

### DETAILED DESCRIPTION OF THE INVENTION

The present invention can be further understood according to the following description.

The present invention describes improved methods for the synthesis of RNA oligonucleotides. In some embodiments, the present invention provides 5′-DMT-2′-Cpep-3′-(N,N-diethyl)cyanoethylphosphoramidites, and methods for their use in oligonucleotides synthesis. These amidites have a significant advantage over other RNA amidites. For example, they utilize 5′-DMT protection, which makes them compatible with conventional amidites and oligomerization processes. The 2′-Cpep protecting group is stable to DMT deprotection and conditions required for phosphoramidite activation during coupling reactions, but can be removed from fully deprotected RNA under acidic conditions that do not facilitate 2′-5′ transesterifcation of the phosphodiester linkages. In addition, the Cpep group does not require orthogonal deprotection, but can be removed in conjunction with the 5′-DMT group following HPLC purification. Furthermore, since the 2′-Cpep RNA is stable to ammonia treatment (unlike 2′-tBDMS), labile protecting groups are not required for the exocyclic amines of the nucleosides. Further, the Cpep group can be incorporated cleanly at the 2′-OH using 5′,3′-TIPS protection, and the monomer is not expensive.

Due to the bulky nature of the Cpep group, coupling rates are slower than for less bulky 2′-protecting groups, which is a detriment for their use in conventional solid phase oligonucleotides synthesis regimes. However, the use of N,N-diethylphosphoramidite provides a significant enhancement in rate of reaction relative to the conventional N,N-diisopropylphosphoramidites. Indeed, such a rate enhancement is critical to efficient coupling of RNA amidites having large 2′-protecting groups on flow-through oligonucleotide synthesizers. While not wishing to be bound by a particular theory, it is believed that the use of such phosphoramidites having less bulky N-substituents, preferably N,N-diisopropylphosphoramidites, provides a rate enhancement that countervails the rate decrease due to the bulk of the Cpep group, thus enabling the practical use of Cpep protected amidites in flow-through oligonucleotide synthesizers.

In addition, The N,N-diethyl phosphoramidite is stable for extended periods when dissolved in organic solvents.

Thus, the present invention provides for tailoring the reactivity of the phosphoramidite to the level of steric hindrance at the 2′-position, due to, for example, a 2′-substituent. Indeed, as with the Cpep group, certain 2′-substituted amidites, such as N,N-diisopropyl MOE amidites, are known to react more slowly than the corresponding deoxy amidites. Accordingly, the use of N,N-dipropyl MOE amidites will improve coupling yields and decrease coupling times.

The present invention provides, in one embodiment, a compound having the formula:

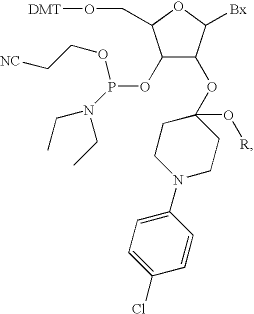

wherein Bx is an optionally protected nucleobase; and R is methyl, ethyl or n-propyl.

In further embodiments, the present invention provides compounds having the formula:

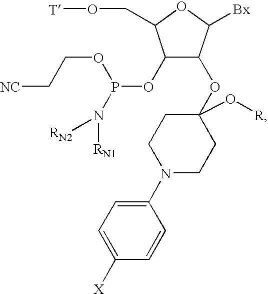

wherein T′ is an acid-labile protecting group; Bx is an optionally protected nucleobase; R is methyl, ethyl, or n-propyl; R_{N1} is H, methyl, ethyl, n-propyl or isopropyl; R_{N2} is, independently of R_{N1} methyl or ethyl; or together R_{N1} and R_{N2} combine to form a pyrrolidinyl, piperidinyl, morpholino or thiomorpholino group; and X is an electron-withdrawing group.

The acid labile protecting group T′ can be any of the many protecting groups suitable for 5′-protection in oligonucleotides synthesis. In some preferred embodiments, T′ is 4,4′-dimethoxytriphenylmethyl or pixyl.

The electron withdrawing group X includes halogens, CN, and other relatively small groups that withdraw electrons either inductively or through resonance effects, as will be immediately apparent to those skilled in the art. In some preferred embodiments, X is F, Cl, Br or CN.

R_{N1} and R_{N2} are preferably selected so that the rate of coupling of the Cpep or modified Cpep amidite is greater than the coupling of the analogous N,N-diisopropyl amidite. Thus, combinations of R_{N1}-R_{N2} having overall small bulk are preferred, such as, without limitation, H-methyl; H-ethyl; H-n-propyl; H-isopropyl; methyl-methyl; methyl-ethyl; methyl-n-propyl; methyl-isopropyl; ethyl-ethyl; ethyl-n-propyl and ethyl-isopropyl. In some preferred embodiments, R_{N1}-R_{N2} are ethyl-ethyl or ethyl-isopropyl; preferably ethyl-ethyl. In some embodiments, RN, and R_{N2} together form a pyrrolidinyl or morpholino moiety.

The present invention also provides processes comprising the steps of:
providing a support-bound species of the formula:

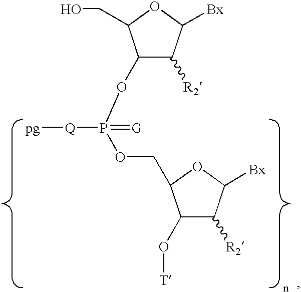

wherein:
n is 0 or a positive integer from 1-100;
each Bx is an optionally protected nucleobase;
each G is O or S;
each Q is O or S;
each pg is H or a protecting group;
each R_{2′} is H, a 2′-deoxy-2′-substituent, or a protected OH group; and
T′ is a support medium or a linker covalently linked to a support medium;
   reacting said support-bound species with an amidite of formula:

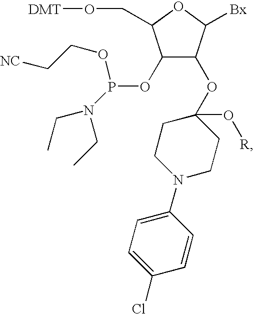

wherein:
Bx is an optionally protected nucleobase;
DMT is the 4,4′-dimethoxytrityl group; and
R is methyl, ethyl or n-propyl;
   to form a support-bound phosphityl compound of formula:

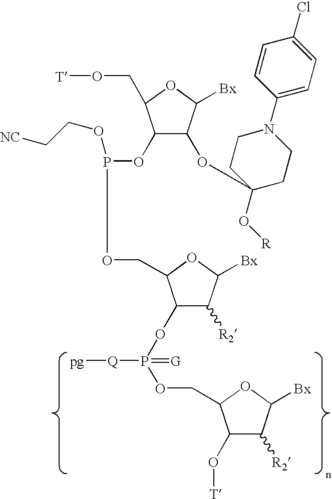

and
(c) oxidizing or sulfurizing the support-bound phosphityl compound to form a phosphotriester compound of formula:

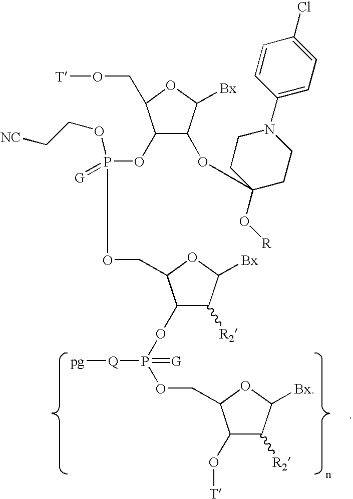

In some embodiments, R is ethyl. In some further embodiments, each Q is O, and each pg is cyanoethyl. In some further embodiments, the process further comprising repeating steps (a)-(c) a plurality of times. In still further embodiments, the process further comprises cleaving the phosphotriester compound from the support medium. In still further embodiments, the process further comprises the step of (d) capping unreacted support bound hydroxyl groups.

In some further embodiments, the present invention provides processes comprising:
(a) providing a support-bound species of the formula:

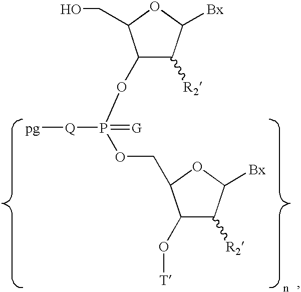

wherein:
   n is 0 or a positive integer from 1 to 100;
   each Bx is an optionally protected nucleobase;
   each G is O or S;
   each Q is O or S;
   each pg is H or a protecting group;
   each R_{2′} is H, a 2′-deoxy-2′-substituent, or a protected OH group; and
   T′ is a support medium or a linker covalently linked to a support medium;
(b) reacting said support-bound species with an amidite of formula:

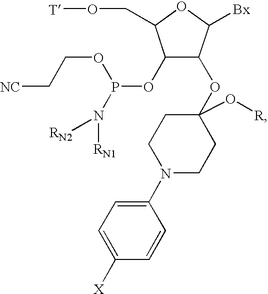

wherein:
   T′ is an acid-labile protecting group;
   Bx is an optionally protected nucleobase;
   R is methyl, ethyl, or n-propyl;
   R_{N1} is H, methyl, ethyl, n-propyl or isopropyl;
   R_{N2} is, independently of R_{N1} methyl or ethyl;
      or together R_{N1} and R_{N2} combine to form a pyrrolidinyl, piperidinyl, morpholino or thiomorpholino group; and
   X is an electron-withdrawing group;
to form a support-bound phosphityl compound of formula:

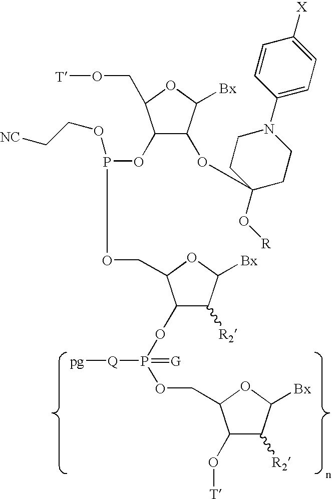

and
oxidizing or sulfurizing the support-bound phosphityl compound to form a phosphotriester compound of formula:

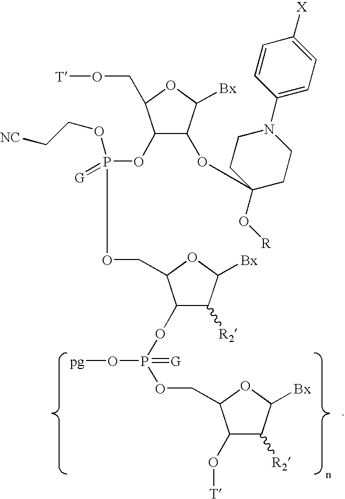

In some embodiments, R is ethyl. In some further embodiments, each Q is O, and each pg is cyanoethyl. In some embodiments, R_{N1} is methyl, ethyl or isopropyl, and R_{N2} is, independently of R_{N1}, methyl or ethyl. In some further embodiments, R_{N1} is methyl and R_{N2} is isopropyl. In some further embodiments, R_{N1} is ethyl and R_{N2} is isopropyl. In some further embodiments, the process further comprises repeating steps (a)-(c) a plurality of times. In still further embodiments, the process further comprises cleaving the phosphotriester compound from the support medium. In still further embodiments, the process further comprises the step of (d) capping unreacted support bound hydroxyl groups.

In the compounds and processes describe herein, the nucleobase Bx is intended to represent any of the nucleobases that occur naturally in genetic material, e.g., A, T, G, C and U, as well as their synthetic analogs as described herein, both with and without nucleobase protecting groups useful in oligonucleotides synthesis. In some embodiments of the compounds and processes of the invention, Bx is U, T or optionally protected G, A, C or 5-methyl C. In further embodiments of the compounds and processes of the invention, Bx is optionally protected G. In further embodiments of the compounds and processes of the invention, Bx is optionally protected A. In further embodiments of the compounds and processes of the invention, Bx is optionally protected C or 5-methyl C. In further embodiments of the compounds and processes of the invention, Bx is U or T. In some embodiments wherein Bx is protected G, Bx is G protected with phenylacetyl. In some embodiments wherein Bx is protected A, Bx is A protected with pivolyl. In some embodiments wherein Bx is protected C or protected 5-methyl C, Bx is C or 5-methyl C protected with phenylacetyl.

As used herein, the term oligonucleotide has the meaning of an oligomer having m subunits embraced within the brackets [ ] of the formula:

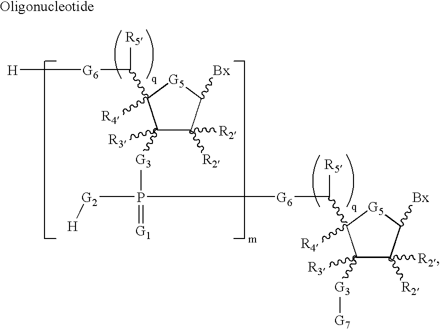

wherein the other variables are defined above, and are described in more detail hereinafter. It is to be understood that, although the oligonucleotide to be made is depicted in a single stranded conformation, it is common for oligonucleotides to be used in a double stranded conformation. For example, in the antisense method referred-to commonly as siRNA, two strands of RNA or RNA-like oligonucleotide are prepared and annealed together, often with a two-nucleotide overlap at the ends. Thus, the present invention contemplates manufacture of both single- and double-stranded oligonucleotides.

### Nucleobases

The nucleobases Bx may be the same or different, and include naturally occurring nucleobases adenine (A), guanine (G), thymine (T), uracil (U) and cytosine (C), as well as modified nucleobases. Modified nucleobases include heterocyclic moieties that are structurally related to the naturally-occurring nucleobases, but which have been chemically modified to impart some property to the modified nucleobase that is not possessed by naturally-occurring nucleobases. The term “nucleobase,” as used herein, is intended to by synonymous with “nucleic acid base or mimetic thereof.” In general, a nucleobase is any substructure that contains one or more atoms or groups of atoms capable of hydrogen bonding to a base of an oligonucleotide.

As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deazaadenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993.

Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; and 5,681,941, certain of which are commonly owned with the instant application, and each of which is herein incorporated by reference, and U.S. Pat. No. 5,750,692, which is commonly owned with the instant application and also herein incorporated by reference.

Additional modifications may also be made at other positions on the oligonucleotide, particularly the 3′ position of the sugar on the 3′ terminal nucleotide and the 5′ position of 5′ terminal nucleotide. For example, one additional modification of the ligand conjugated oligonucleotides of the present invention involves chemically linking to the oligonucleotide one or more additional non-ligand moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA, 1989, 86, 6553), cholic acid (Manoharan et al., Bioorg. Med. Chem. Lett., 1994, 4, 1053), a thioether, e.g., hexyl-S-tritylthiol (Manoharan et al., Ann. N.Y. Acad. Sci., 1992, 660, 306; Manoharan et al., Bioorg. Med. Chem. Let., 1993, 3, 2765), a thiocholesterol (Oberhauser et al., Nucl. Acids Res., 1992, 20, 533), an aliphatic chain, e.g., dodecandiol or undecyl residues (Saison-Behmoaras et al., EMBO J., 1991, 10, 111; Kabanov et al., FEBS Lett., 1990, 259, 327; Svinarchuk et al., Biochimie, 1993, 75, 49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651; Shea et al., Nucl. Acids Res., 1990, 18, 3777), a polyamine or a polyethylene glycol chain (Manoharan et al., Nucleosides & Nucleotides, 1995, 14, 969), or adamantane acetic acid (Manoharan et al., Tetrahedron Lett., 1995, 36, 3651), a palmityl moiety (Mishra et al., Biochim. Biophys. Acta, 1995, 1264, 229), or an octadecylamine or hexylamino-carbonyl-oxycholesterol moiety (Crooke et al., J. Pharmacol. Exp. Ther., 1996, 277, 923).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned, and each of which is herein incorporated by reference.

In some embodiments of the invention, oligomeric compounds, e.g. oligonucleotides, are prepared having polycyclic heterocyclic compounds in place of one or more heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

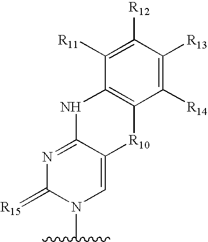

Representative cytosine analogs that make 3 hydrogen bonds with a guanosine in a second strand include 1,3-diazaphenoxazine-2-one (R₁₀₌O, R₁₁-R₁₄=H) [Kurchavov, et al., Nucleosides and Nucleotides, 1997, 16, 1837-1846], 1,3-diazaphenothiazine-2-one (R₁₀=S, R₁₁-R₁₄=H), [Lin, K. -Y.; Jones, R. J.; Matteucci, M. J. Am. Chem. Soc. 1995, 117, 3873-3874] and 6,7,8,9-tetrafluoro-1,3-diazaphenoxazine-2-one (R₁₀=O, R₁₁-R₁₄=F) [Wang, J.; Lin, K. -Y., Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions (also see U.S. patent application entitled “Modified Peptide Nucleic Acids” filed May 24, 2002, Ser. No. 10/155,920; and U.S. patent application entitled “Nuclease Resistant Chimeric Oligonucleotides” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀₌O, R₁₁=—O—(CH₂)₂—NH₂, R₁₂₋₁₄=H) [Lin, K. -Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° relative to 5-methyl cytosine (dC5^{me}), which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183, which issued on May 22, 2000, and U.S. Pat. No. 6,007,992, which issued on Dec. 28, 1999, the contents of both are commonly assigned with this application and are incorporated herein in their entirety. Such compounds include those having the formula:

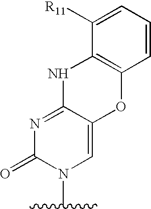

Wherein R₁₁ includes (CH₃)₂N—(CH₂)₂—O—; H₂N—(CH₂)₃—; Ph-CH₂—O—C(═O)—N(H)—(CH₂)₃—; H₂N—; Fluorenyl-CH₂—O—C(═O)—N(H)—(CH₂)₃—; Phthalimidyl-CH₂—O—C(═O)—N(H)—(CH₂)₃—; Ph-CH₂—O—C(═O)—N(H)—(CH₂)₂—O—; Ph-CH₂—O—C(═O)—N(H)—(CH₂)₃—O—; (CH₃)₂N—N(H)—(CH₂)₂—O—; Fluorenyl-CH₂—O—C(═O)—N(H)—(CH₂)₂—O—; Fluorenyl-CH₂—O—C(═O)—N(H)—(CH₂)₃—O—; H₂N—(CH₂)₂—O—CH₂—; N₃—(CH₂)₂—O—CH₂—; H₂N—(CH₂)₂—O—, and NH₂C(═NH)NH—.

Also disclosed are tricyclic heterocyclic compounds of the formula:

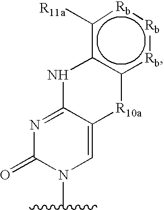

wherein:
R₁₀ₐ is O, S or N—CH₃; R₁₁ₐ is A(Z)ₓ₁, wherein A is a spacer and Z independently is a label bonding group bonding group optionally bonded to a detectable label, but R₁₁ₐ is not amine, protected amine, nitro or cyano; X1 is 1, 2 or 3; and R_{b} is independently —CH═, —N═, —C(C₁₋₈ alkyl)═ or —C(halogen)═, but no adjacent R_{b} are both —N═, or two adjacent R_{b} are taken together to form a ring having the structure:

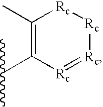

where R_{c} is independently —CH═, —N═, —C(C₁₋₈ alkyl)═ or —C(halogen)═, but no adjacent R_{b} are both —N═.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K. -Y.; Matteucci, M. J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W. M.; Wolf, J. J.; Olson, P.; Grant, D.; Lin, K. -Y.; Wagner, R. W.; Matteucci, M. Proc. Natl. Acad. Sci. USA, 1999, 96, 3513-3518]. Nevertheless, to optimize oligonucleotide design and to better understand the impact of these heterocyclic modifications on the biological activity, it is important to evaluate their effect on the nuclease stability of the oligomers.

Further tricyclic and tetracyclic heteroaryl compounds amenable to the present invention include those having the formulas:

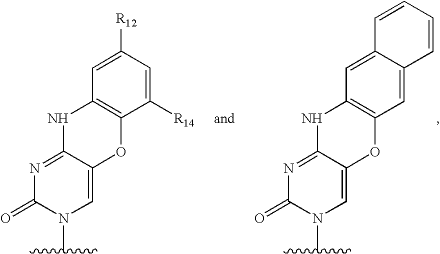

wherein R₁₄ is NO₂ or both R₁₄ and R₁₂ are independently —CH₃. The synthesis of these compounds is disclosed in U.S. Pat. No. 5,434,257, which issued on Jul. 18, 1995, U.S. Pat. No. 5,502,177, which issued on Mar. 26, 1996, and U.S. Pat. No. 5,646,269, which issued on Jul. 8, 1997, the contents of which are commonly assigned with this application and are incorporated herein in their entirety.

Further tricyclic heterocyclic compounds amenable to the present invention also disclosed in the “257, 177 and 269” Patents include those having the formula:

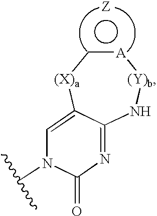

wherein a and b are independently 0 or 1 with the total of a and b being 0 or 1; A is N, C or CH; X is S, O, C═O, NH or NCH₂, R⁶; Y is C═O; Z is taken together with A to form an aryl or heteroaryl ring structure comprising 5 or 6 ring atoms wherein the heteroaryl ring comprises a single O ring heteroatom, a single N ring heteroatom, a single S ring heteroatom, a single O and a single N ring heteroatom separated by a carbon atom, a single S and a single N ring heteroatom separated by a C atom, 2 N ring heteroatoms separated by a carbon atom, or 3 N ring heteroatoms at least 2 of which are separated by a carbon atom, and wherein the aryl or heteroaryl ring carbon atoms are unsubstituted with other than H or at least 1 nonbridging ring carbon atom is substituted with R²⁰ or ═O; or Z is taken together with A to form an aryl ring structure comprising 6 ring atoms wherein the aryl ring carbon atoms are unsubstituted with other than H or at least 1 nonbridging ring carbon atom is substituted with R⁶ or ═O; R⁶ is independently H, C₁₋₆ alkyl, C₂₋₆ alkenyl, C₂₋₆ alkynyl, NO₂, N(R³)₂, CN or halo, or an R⁶ is taken together with an adjacent Z group R⁶ to complete a phenyl ring; R²⁰ is, independently, H, C₁₋₆ alkyl, C₂₋₆ alkyl, C₂₋₆ alkenyl, C₂₋₆ alkynyl, NO₂, N(R²¹)₂, CN, or halo, or an R²⁰ is taken together with an adjacent R²⁰ to complete a ring containing 5 or 6 ring atoms, and tautomers, solvates and salts thereof; R²¹ is, independently, H or a protecting group; R³ is a protecting group or H; and tautomers, solvates and salts thereof.

More specific examples of bases included in the “257, 177 and 269” Patents are compounds of the formula:

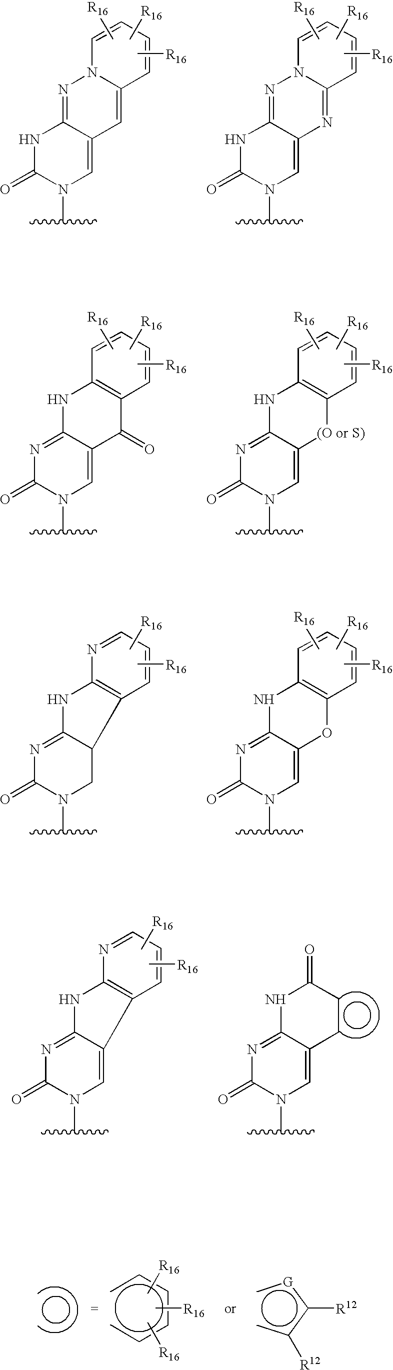

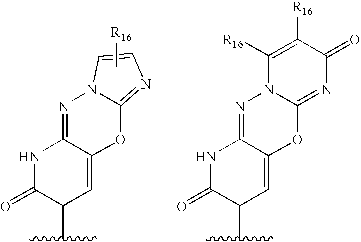

wherein each R₁₆, is, independently, selected from hydrogen and various substituent groups.

Further polycyclic base moieties having the formula:

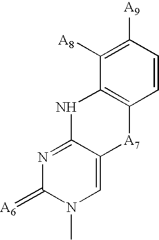

wherein: A₆ is O or S; A₇ is CH₂, N—CH₃, O or S; each A₈ and A₉ is hydrogen or one of A₈ and A₉ is hydrogen and the other of A₈ and A₉ is selected from the group consisting of:

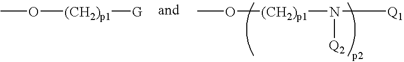

wherein: G is —CN, —OA₁₀, —SA₁₀, —N(H)A₁₀, —ON(H)A₁₀ or —C(═NH)N(H)A₁₀; Q₁ is H, —NHA₁₀, —C(═O)N(H)A₁₀, —C(═S)N(H)A₁₀ or —C(═NH)N(H)A₁₀; each Q₂ is, independently, H or Pg; A₁₀ is H, Pg, substituted or unsubstituted C₁-C₁₀ alkyl, acetyl, benzyl, —(CH₂)ₚ₃NH₂, —(CH₂)ₚ₃N(H)Pg, a D or L α-amino acid, or a peptide derived from D, L or racemic α-amino acids; Pg is a nitrogen, oxygen or thiol protecting group; each p1 is, independently, from 2 to about 6; p2 is from 1 to about 3; and p3 is from 1 to about 4; are disclosed in U.S. patent application Ser. No. 09/996,292 filed Nov. 28, 2001, which is commonly owned with the instant application, and is herein incorporated by reference.

### Sugars and Sugar Substituents

The sugar moiety:

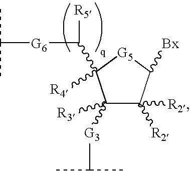

wherein each dashed line [symbol] indicates a point of attachment to an adjacent phosphorus atom, represents the sugar portion of a general nucleoside or nucleotide as embraced by the present invention.

Suitable 2′-substituents corresponding to R′₂ include: OH, F, O-alkyl (e.g. O-methyl), S-alkyl, N-alkyl, O-alkenyl, S-alkenyl, N-alkenyl; O-alkynyl, S-alkynyl, N-alkynyl; O-alkyl-O-alkyl, wherein the alkyl, alkenyl and alkynyl may be substituted or unsubstituted C₁ to C₁₀ alkyl or C₂ to C₁₀ alkenyl or alkynyl, respectively. Particularly preferred are O[(CH₂)_{g}O]ₕCH₃, O(CH₂)_{g}OCH₃, O(CH₂)_{g}NH₂, O(CH₂)_{g}CH₃, O(CH₂)_{g}ONH₂, and O(CH₂)_{g}ON[(CH₂)_{g}CH₃]₂, where g and h are from 1 to about 10. Other preferred oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred 2′-modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504). A further preferred modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples hereinbelow.

Other preferred modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. A preferred 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Further representative substituent groups include groups of formula Iₐ or IIₐ:

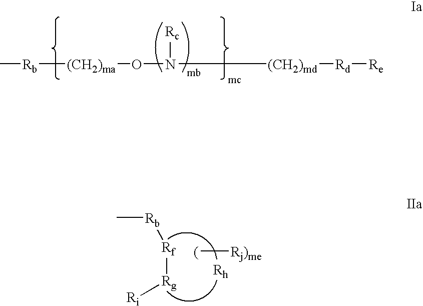

wherein: R_{b} is O, S or NH; R_{d} is a single bond, O or C(═O); Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIₐ;

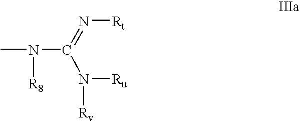

Each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl; or optionally, Rᵤ and Rᵥ, together form a phthalimido moiety with the nitrogen atom to which they are attached; each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl; Rₖ is hydrogen, a nitrogen protecting group or —Rₓ—R_{y}; Rₚ is hydrogen, a nitrogen protecting group or —Rₓ—R_{y}; Rₓ is a bond or a linking moiety; R_{y} is a chemical functional group, a conjugate group or a solid support medium medium; each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ), guanidino and acyl where said acyl is an acid amide or an ester; or Rₘ and Rₙ, together, are a nitrogen protecting group, are joined in a ring structure that optionally includes an additional heteroatom selected from N and O or are a chemical functional group; Rᵢ is OR_{z}, SR_{z}, or N(R_{z})₂; each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ; R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN; mₐ is 1 to about 10; each mb is, independently, 0 or 1; mc is 0 or an integer from 1 to 10; md is an integer from 1 to 10; me is from 0, 1 or 2; and provided that when mc is 0, md is greater than 1.

Representative substituents groups of Formula I are disclosed in U.S. Pat. No. 6,172,209. Representative cyclic substituent groups of Formula II are disclosed in U.S. Pat. No. 6,271,358.

Particularly useful sugar substituent groups include O[(CH₂)_{g}O]ₕCH₃, O(CH₂)_{g}OCH₃, O(CH₂)_{g}NH₂, O(CH₂)_{g}CH₃, O(CH₂)_{g}ONH₂, and O(CH₂)_{g}ON[(CH₂)_{g}CH₃)]₂, where g and h are from 1 to about 10.

Some particularly useful oligomeric compounds of the invention contain at least one nucleoside having one of the following substituent groups: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligomeric compound, or a group for improving the pharmacodynamic properties of an oligomeric compound, and other substituents having similar properties. A preferred modification includes 2′-methoxyethoxy [2′-O—CH₂CH₂OCH₃, also known as 2′-O-(2-methoxyethyl) or 2′-MOE] (Martin et al., Helv. Chim. Acta, 1995, 78, 486), i.e., an alkoxyalkoxy group. A further preferred modification is 2′-dimethylaminooxyethoxy, i.e., a O(CH₂)₂ON(CH₃)₂ group, also known as 2′-DMAOE. Representative aminooxy substituent groups are described in co-owned U.S. patent application Ser. No. 09/344,260, filed Jun. 25, 1999, entitled “Aminooxy-Functionalized Oligomers”; and U.S. patent application Ser. No. 09/370,541, filed Aug. 9, 1999, entitled “Aminooxy-Functionalized Oligomers and Methods for Making Same;” hereby incorporated by reference in their entirety.

Other particularly advantageous 2′-modifications include 2′-methoxy (2′-O—CH₃), 2′-aminopropoxy (2′-OCH₂CH₂CH₂NH₂) and 2′-fluoro (2′-F). Similar modifications may also be made at other positions on nucleosides and oligomers, particularly the 3′ position of the sugar on the 3′ terminal nucleoside or at a 3′-position of a nucleoside that has a linkage from the 2′-position such as a 2′-5′ linked oligomer and at the 5′ position of a 5′ terminal nucleoside. Oligomers may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,0531 5,639,873; 5,646,265; 5,658,873; 5,670,633; and 5,700,920, certain of which are commonly owned, and each of which is herein incorporated by reference, and commonly owned U.S. patent application Ser. No. 08/468,037, filed on Jun. 5, 1995, also herein incorporated by reference.

Representative guanidino substituent groups that are shown in formula III and IV are disclosed in co-owned U.S. patent application Ser. No. 09/349,040, entitled “Functionalized Oligomers”, filed Jul. 7, 1999, issue fee paid on Oct. 23, 2002.

Representative acetamido substituent groups are disclosed in U.S. Pat. No. 6,147,200 which is hereby incorporated by reference in its entirety. Representative dimethylaminoethyloxyethyl substituent groups are disclosed in International Patent Application PCT/US99/17895, entitled “2′-O-Dimethylaminoethyloxyethyl-Modified Oligonucleotides”, filed Aug. 6, 1999, hereby incorporated by reference in its entirety. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be joined to form a circular structure by hybridization or by formation of a covalent bond, however, open linear structures are generally preferred. Within the oligonucleotide structure, the phosphate groups are commonly referred to as forming the internucleoside linkages of the oligonucleotide. The normal internucleoside linkage of RNA and DNA is a 3′ to 5′ phosphodiester linkage.

While the present invention may be adapted to produce oligonucleotides for any desired end use (e.g. as probes for us in the polymerase chain reaction), one preferred use of the oligonucleotides is in antisense therapeutics. One mode of action that is often employed in antisense therapeutics is the so-called RNAse H mechanism, whereby a strand of DNA is introduced into a cell, where the DNA hybridizes to a strand of RNA. The DNA-RNA hybrid is recognized by an endonuclease, RNAse H, which cleaves the RNA strand. In normal cases, the RNA strand is messenger RNA (mRNA), which, after it has been cleaved, cannot be translated into the corresponding peptide or protein sequence in the ribosomes. In this way, DNA may be employed as an agent for modulating the expression of certain genes.

It has been found that by incorporating short stretches of DNA into an oligonucleotide, the RNAse H mechanism can be effectively used to modulate expression of target peptides or proteins. In some embodiments of the invention, an oligonucleotide incorporating a stretch of DNA and a stretch of RNA or 2′-modified RNA can be used to effectively modulate gene expression. In preferred embodiments, the oligonucleotide comprises a stretch of DNA flanked by two stretches of 2′-modified RNA. Preferred 2′-modifications include 2′-MOE as described herein.

The ribosyl sugar moiety has also been extensively studied to evaluate the effect its modification has on the properties of oligonucleotides relative to unmodified oligonucleotides. The 2′-position of the sugar moiety is one of the most studied sites for modification. Certain 2′-substituent groups have been shown to increase the lipohpilicity and enhance properties such as binding affinity to target RNA, chemical stability and nuclease resistance of oligonucleotides. Many of the modifications at the 2′-position that show enhanced binding affinity also force the sugar ring into the C₃-endo conformation.

RNA exists in what has been termed “A Form” geometry while DNA exists in “B Form” geometry. In general, RNA:RNA duplexes are more stable, or have higher melting temperatures (Tm) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). In addition, the 2′ hydroxyl groups of RNA can form a network of water mediated hydrogen bonds that help stabilize the RNA duplex (Egli et al., Biochemistry, 1996, 35, 8489-8494).

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of a DNA:RNA hybrid is central to antisense therapies as the mechanism requires the binding of a modified DNA strand to a mRNA strand. To effectively inhibit the mRNA, the antisense DNA should have a very high binding affinity with the mRNA. Otherwise the desired interaction between the DNA and target mRNA strand will occur infrequently, thereby decreasing the efficacy of the antisense oligonucleotide.

Various synthetic modifications have been proposed to increase nuclease resistance, or to enhance the affinity of the antisense strand for its target mRNA (Crooke et al., Med. Res. Rev., 1996, 16, 319-344; De Mesmaeker et al., Acc. Chem. Res., 1995, 28, 366-374). A variety of modified phosphorus-containing linkages have been studied as replacements for the natural, readily cleaved phosphodiester linkage in oligonucleotides. In general, most of them, such as the phosphorothioate, phosphoramidates, phosphonates and phosphorodithioates all result in oligonucleotides with reduced binding to complementary targets and decreased hybrid stability.

RNA exists in what has been termed “A Form” geometry while DNA exists in “B Form” geometry. In general, RNA:RNA duplexes are more stable, or have higher melting temperatures (Tm) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2=hydroxyl in RNA biases the sugar toward a C3=endo pucker, i.e., also designated as Northern pucker, which causes the duplex to favor the A-form geometry. On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). In addition, the 2=hydroxyl groups of RNA can form a network of water mediated hydrogen bonds that help stabilize the RNA duplex (Egli et al., Biochemistry, 1996, 35, 8489-8494).

DNA:RNA hybrid duplexes, however, are usually less stable than pure RNA:RNA duplexes and, depending on their sequence, may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of a DNA:RNA hybrid a significant aspect of antisense therapies, as the proposed mechanism requires the binding of a modified DNA strand to a mRNA strand. Ideally, the antisense DNA should have a very high binding affinity with the mRNA. Otherwise, the desired interaction between the DNA and target mRNA strand will occur infrequently, thereby decreasing the efficacy of the antisense oligonucleotide.

One synthetic 2′-modification that imparts increased nuclease resistance and a very high binding affinity to nucleotides is the 2=-methoxyethoxy (MOE, 2′-OCH₂CH₂OCH₃) side chain (Baker et al., J. Biol. Chem., 1997, 272, 11944-12000; Freier et al., Nucleic Acids Res., 1997, 25, 4429-4443). One of the immediate advantages of the MOE substitution is the improvement in binding affinity, which is greater than many similar 2′ modifications such as O-methyl, O-propyl, and O-aminopropyl (Freier and Altmann, Nucleic Acids Research, (1997) 25:4429-4443). 2=-O-Methoxyethyl-substituted oligonucleotides also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, they display improved RNA affinity and higher nuclease resistance. Chimeric oligonucleotides with 2=-O-methoxyethyl-ribonucleoside wings and a central DNA-phosphorothioate window also have been shown to effectively reduce the growth of tumors in animal models at low doses. MOE substituted oligonucleotides have shown outstanding promise as antisense agents in several disease states. One such MOE substituted oligonucleotide is presently being investigated in clinical trials for the treatment of CMV retinitis.

LNAs (oligonucleotides wherein the 2′ and 4′ positions are connected by a bridge) also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

LNAs in which the 2′-hydroxyl group is linked to the 4′ carbon atom of the sugar ring thereby forming a 2′-C,4′-C-oxymethylene linkage thereby forming a bicyclic sugar moiety. The linkage may be a methylene (—CH₂—)ₙ group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10 C), stability towards 3′-exonucleolytic degradation and good solubility properties. Other preferred bridge groups include the 2′-deoxy-2′-CH₂OCH₂-4′ bridge.

### Alternative Linkers

In addition to phosphate diester and phosphorothioate diester linkages, other linkers are known in the art. While the primary concern of the present invention has to do with phosphate diester and phosphorothioate diester oligonucleotides, chimeric compounds having more than one type of linkage, as well as oligomers having non-phosphate/phosphorothioate diester linkages as described in further detail below, are also contemplated in whole or in part within the context of the present invention.

Exemplary non-phosphate/phosphorothioate diester linkages contemplated within the skill of the art include: phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates. Additional linkages include: thiodiester (—O—C(O)—S—), thionocarbamate (—O—C(O)(NJ)-S—), siloxane (—O—Si(J)₂-O—), carbamate (—O—C(O)—NH— and —NH—C(O)—O—), sulfamate (—O—S(O)(O)—N— and —N—S(O)(O)—N—, morpholino sulfamide (—O—S(O)(N(morpholino)-), sulfonamide (—O—SO₂—NH—), sulfide (—CH₂—S—CH₂—), sulfonate (—O—SO₂—CH₂—), N,N′-dimethylhydrazine (—CH₂—N(CH₃)—N(CH₃)—), thioformacetal (—S—CH₂—O—), formacetal (—O—CH₂—O—), thioketal (—S—C(J)₂—O—), ketal (—O—C(J)₂-O—), amine (—NH—CH₂—CH₂—), hydroxylamine (—CH₂—N(J)-O—), hydroxylimine (—CH═N—O—), and hydrazinyl (—CH₂—N(H)—N(H)—).

In each of the foregoing substructures relating to internucleoside linkages, J denotes a substituent group which is commonly hydrogen or an alkyl group or a more complicated group that varies from one type of linkage to another.

In addition to linking groups as described above that involve the modification or substitution of the —O—P—O— atoms of a naturally occurring linkage, included within the scope of the present invention are linking groups that include modification of the 5′-methylene group as well as one or more of the —O—P—O— atoms. Linkages of this type are well documented in the prior art and include without limitation the following: amides (—CH₂—CH₂—N(H)—C(O)) and —CH₂—O—N═CH—; and alkylphosphorus (—C(J)₂-P(═O)(OJ)-C(J)₂-C(J)₂-). J is as described above.

### Oligonucleotide Synthesis

Oligonucleotides are generally prepared, as described above, on a support medium, e.g. a solid support medium. In general a first synthon (e.g. a monomer, such as a nucleoside) is first attached to a support medium, and the oligonucleotide is then synthesized by sequentially coupling monomers to the support-bound synthon. This iterative elongation eventually results in a final oligomeric compound or other polymer such as a polypeptide. Suitable support medium can be soluble or insoluble, or may possess variable solubility in different solvents to allow the growing support bound polymer to be either in or out of solution as desired. Traditional support medium such as solid support media are for the most part insoluble and are routinely placed in reaction vessels while reagents and solvents react with and/or wash the growing chain until the oligomer has reached the target length, after which it is cleaved from the support and, if necessary further worked up to produce the final polymeric compound. More recent approaches have introduced soluble supports including soluble polymer supports to allow precipitating and dissolving the iteratively synthesized product at desired points in the synthesis (Gravert et al., Chem. Rev., 1997, 97, 489-510).

The term support medium is intended to include all forms of support known to the art skilled for the synthesis of oligomeric compounds and related compounds such as peptides. Some representative support medium that are amenable to the methods of the present invention include but are not limited to the following: controlled pore glass (CPG); oxalyl-controlled pore glass (see, e.g., Alul, et al., Nucleic Acids Research 1991, 19, 1527); silica-containing particles, such as porous glass beads and silica gel such as that formed by the reaction of trichloro-[3-(4-chloromethyl)phenyl]propylsilane and porous glass beads (see Parr and Grohmann, Angew. Chem. Internal. Ed. 1972, 11, 314, sold under the trademark “PORASIL E” by Waters Associates, Framingham, Mass., USA); the mono ester of 1,4-dihydroxymethylbenzene and silica (see Bayer and Jung, Tetrahedron Lett., 1970, 4503, sold under the trademark “BIOPAK” by Waters Associates); TENTAGEL (see, e.g., Wright, et al., Tetrahedron Letters 1993, 34, 3373); cross-linked styrene/divinylbenzene copolymer beaded matrix or POROS, a copolymer of polystyrene/divinylbenzene (available from Perceptive Biosystems); soluble support medium, polyethylene glycol PEG's (see Bonora et al., Organic Process Research & Development, 2000, 4, 225-231).

Further support medium amenable to the present invention include without limitation PEPS support a polyethylene (PE) film with pendant long-chain polystyrene (PS) grafts (molecular weight on the order of 10⁶, (see Berg, et al., J. Am. Chem. Soc., 1989, 111, 8024 and International Patent Application WO 90/02749),). The loading capacity of the film is as high as that of a beaded matrix with the additional flexibility to accommodate multiple syntheses simultaneously. The PEPS film may be fashioned in the form of discrete, labeled sheets, each serving as an individual compartment. During all the identical steps of the synthetic cycles, the sheets are kept together in a single reaction vessel to permit concurrent preparation of a multitude of peptides at a rate close to that of a single peptide by conventional methods. Also, experiments with other geometries of the PEPS polymer such as, for example, non-woven felt, knitted net, sticks or microwellplates have not indicated any limitations of the synthetic efficacy.

Further support medium amenable to the present invention include without limitation particles based upon copolymers of dimethylacrylamide cross-linked with N,N′-bisacryloylethylenediamine, including a known amount of N-tertbutoxycarbonyl-beta-alanyl-N′-acryloylhexamethylenediamine. Several spacer molecules are typically added via the beta alanyl group, followed thereafter by the amino acid residue subunits. Also, the beta alanyl-containing monomer can be replaced with an acryloyl safcosine monomer during polymerization to form resin beads. The polymerization is followed by reaction of the beads with ethylenediamine to form resin particles that contain primary amines as the covalently linked functionality. The polyacrylamide-based supports are relatively more hydrophilic than are the polystyrene-based supports and are usually used with polar aprotic solvents including dimethylformamide, dimethylacetamide, N-methylpyrrolidone and the like (see Atherton, et al., J. Am. Chem. Soc., 1975, 97, 6584, Bioorg Chem. 1979, 8, 351, and J. C. S. Perkin I 538 (1981)).

Further support medium amenable to the present invention include without limitation a composite of a resin and another material that is also substantially inert to the organic synthesis reaction conditions employed. One exemplary composite (see Scott, et al., J. Chrom. Sci., 1971, 9, 577) utilizes glass particles coated with a hydrophobic, cross-linked styrene polymer containing reactive chloromethyl groups, and is supplied by Northgate Laboratories, Inc., of Hamden, Conn., USA. Another exemplary composite contains a core of fluorinated ethylene polymer onto which has been grafted polystyrene (see Kent and Merrifield, Israel J. Chem. 1978, 17, 243 and van Rietschoten in Peptides 1974, Y. Wolman, Ed., Wiley and Sons, New York, 1975, pp. 113-116). Contiguous solid support media other than PEPS, such as cotton sheets (Lebl and Eichler, Peptide Res. 1989, 2, 232) and hydroxypropylacrylate-coated polypropylene membranes (Daniels, et al., Tetrahedron Lett. 1989, 4345). Acrylic acid-grafted polyethylene-rods and 96-microtiter wells to immobilize the growing peptide chains and to perform the compartmentalized synthesis. (Geysen, et al., Proc. Natl. Acad. Sci. USA, 1984, 81, 3998). A “tea bag” containing traditionally-used polymer beads. (Houghten, Proc. Natl. Acad. Sci. USA, 1985, 82, 5131). Simultaneous use of two different supports with different densities (Tregear, Chemistry and Biology of Peptides, J. Meienhofer, ed., Ann Arbor Sci. Publ., Ann Arbor, 1972 pp. 175-178). Combining of reaction vessels via a manifold (Gorman, Anal. Biochem., 1984, 136, 397). Multicolumn solid-phase synthesis (e.g., Krchnak, et al., Int. J. Peptide Protein Res., 1989, 33, 209), and Holm and Meldal, in “Proceedings of the 20th European Peptide Symposium”, G. Jung and E. Bayer, eds., Walter de Gruyter & Co., Berlin, 1989 pp. 208-210). Cellulose paper (Eichler, et al., Collect. Czech. Chem. Commun., 1989, 54, 1746). Support mediumted synthesis of peptides have also been reported (see, Synthetic Peptides: A User's Guide, Gregory A. Grant, Ed. Oxford University Press 1992; U.S. Pat. Nos. 4,415,732; 4,458,066; 4,500,707; 4,668,777; 4,973,679; 5,132,418; 4,725,677 and Re-34,069.)

Support bound oligonucleotide synthesis relies on sequential addition of nucleotides to one end of a growing chain. Typically, a first nucleoside (having protecting groups on any exocyclic amine functionalities present) is attached to an appropriate glass bead support and activated phosphite compounds (typically nucleotide phosphoramidites, also bearing appropriate protecting groups) are added stepwise to elongate the growing oligonucleotide. Additional methods for solid-phase synthesis may be found in Caruthers U.S. Pat. Nos. 4,415,732; 4,458,066; 4,500,707; 4,668,777; 4,973,679; and 5,132,418; and Koster U.S. Pat. Nos. 4,725,677 and Re. 34,069.

Commercially available equipment routinely used for the support medium based synthesis of oligomeric compounds and related compounds is sold by several vendors including, for example, Applied Biosystems (Foster City, Calif.). Any other means for such synthesis known in the art may additionally or alternatively be employed. Suitable solid phase techniques, including automated synthesis techniques, are described in F. Eckstein (ed.), Oligonucleotides and Analogues, a Practical Approach, Oxford University Press, New York (1991).

In general, the phosphorus protecting group (pg) is an alkoxy or alkylthio group or O or S having a β-eliminable group of the formula —CH₂CH₂-G_{w}, wherein G, is an electron-withdrawing group. Suitable examples of pg that are amenable to use in connection with the present invention include those set forth in the Caruthers U.S. Pat. Nos. 4,415,732; 4,458,066; 4,500,707; 4,668,777; 4,973,679; and 5,132,418; and Köster U.S. Pat. Nos. 4,725,677 and Re. 34,069. In general the alkyl or cyanoethyl withdrawing groups are preferred, as commercially available phosphoramidites generally incorporate either the methyl or cyanoethyl phosphorus protecting group.

The method for removal of pg depends upon the specific pg to be removed. The β-eliminable groups, such as those disclosed in the Köster et al. patents, are generally removed in a weak base solution, whereby an acidic β-hydrogen is extracted and the —CH₂CH₂-G_{w} group is eliminated by rearrangement to form the corresponding acrylo-compound CH₂═CH-G_{w}. In contrast, an alkyl group is generally removed by nucleophilic attack on the a-carbon of the alkyl group. Such PGs are described in the Caruthers et al. patents, as cited herein.

The person skilled in the art will recognize that oxidation of P(III) to P(V) can be carried out by a variety of reagents. Furthermore, the person skilled in the art will recognize that the P(V) species can exist as phosphate triesters, phosphorothioate diesters, or phosphorodithioate diesters. Each type of P(V) linkage has uses and advantages, as described herein. Thus, the term “oxidizing agent” should be understood broadly as being any reagent capable of transforming a P(III) species (e.g. a phosphite) into a P(V) species. Thus the term “oxidizing agent” includes “sulfurizing agent,” which is also considered to have the same meaning as “thiation reagent.” Oxidation, unless otherwise modified, indicates introduction of oxygen or sulfur, with a concomitant increase in P oxidation state from III to V. Where it is important to indicate that an oxidizing agent introduces an oxygen into a P(III) species to make a P(V) species, the oxidizing agent will be referred to herein is “an oxygen-introducing oxidizing reagent.”

Oxidizing reagents for making phosphate diester linkages (i.e. oxygen-introducing oxidizing reagents) under the phosphoramidite protocol have been described by e.g. Caruthers et al. and Köster et al., as cited herein. Examples of sulfurization reagents which have been used to synthesize oligonucleotides containing phosphorothioate bonds include elemental sulfur, dibenzoyltetrasulfide, 3-H-1,2-benzidithiol-3-one 1,1-dioxide (also known as Beaucage reagent), tetraethylthiuram disulfide (TETD), and bis(O,O-diisopropoxy phosphinothioyl) disulfide (known as Stec reagent). Oxidizing reagents for making phosphorothioate diester linkages include phenylacetyldisulfide (PADS), as described by Cole et al. in U.S. Pat. No. 6,242,591. In some embodiments of the invention, the phosphorothioate diester and phosphate diester linkages may alternate between sugar subunits. In other embodiments of the present invention, phosphorothioate linkages alone may be employed. In some embodiments, the thiation reagent may be a dithiuram disulfides. See U.S. Pat. No. 5,166,387 for disclosure of some suitable dithiuram disulfides. It has been surprisingly found that one dithiuram disulfide may be used together with a standard capping reagent, so that capping and oxidation may be conducted in the same step. This is in contrast to standard oxidative reagents, such as Beaucage reagent, which require that capping and oxidation take place in separate steps, generally including a column wash between steps.

The 5′-protecting group bg or T′ is a protecting group that is orthogonal to the protecting groups used to protect the nucleobases, and is also orthogonal, where appropriate to 2′-O-protecting groups, as well as to the 3′-linker to the solid support medium. In some embodiments of the invention, the 5′-protecting group is acid labile. In some embodiments according to the invention, the 5′-protecting group is selected from an optionally substituted trityl group and an optionally substituted pixyl group. In some embodiments, the pixyl group is substituted with one or more substituents selected from alkyl, alkoxy, halo, alkenyl and alkynyl groups. In some embodiments, the trityl groups are substituted with from about 1 to about 3 alkoxy groups, specifically about 1 to about 3 methoxy groups. In particular embodiments of the invention, the trityl groups are substituted with 1 or 2 methoxy groups at the 4- and (if applicable) 4′-positions. A particularly acceptable trityl group is 4,4′-dimethoxytrityl (DMT or DMTr).

In the context of the present invention, the term “reagent push” has the meaning of a volume of solvent that is substantially free of any active compound (i.e. reagent, activator, by-product, or other substance other than solvent), which volume of solvent is introduced to the column for the purpose, and with the effect, of pushing a reagent solution onto and through the column ahead of a subsequent reagent solution. A reagent push need not be an entire column volume, although in some cases it may include one or more column volumes. In some embodiments, a reagent push, comprises at least the minimum volume necessary to substantially clear reagent, by-products and/or activator from a cross-section of the column immediately ahead of the front formed by the reagent solution used for the immediately subsequent synthetic step. An active compound, whether a reagent, by-product or activator, is considered substantially cleared if the concentration of the compound in a cross-section of the column at which the following reagent solution front is located, is low enough that it does not substantially affect the activity of the following reagent solution. The person skilled in the art will recognize that this the volume of solvent required for a “reagent push” will vary depending upon the solvent, the solubility in the solvent of the reagents, activators, by-products, etc., that are on the column, the amounts of reagents, activators, by-products, etc. that are to be cleared from the column, etc. It is considered within the skill of the artisan to select an appropriate volume for each reagent push, especially with an eye toward the Examples, below.

As used herein, unless “column wash” is otherwise modified, it has the same meaning as “reagent push.” In some embodiments of the invention, column wash may imply that at least one column volume is permitted to pass through the column before the subsequent reagent solution is applied to the column. Where a column volume (CV) of the column wash is specified, this indicates that a volume of solvent equivalent to the interior volume of the unpacked column is used for the column wash.

In the context of the present invention, a wash solvent is a solvent containing substantially no active compound that is applied to a column between synthetic steps. A “wash step” is a step in which a wash solvent is applied to the column. Both “reagent push” and “column wash” are included within this definition of “wash step”.

A wash solvent may be a pure chemical compound or a mixture of chemical compounds, the solvent being capable of dissolving an active compound.

In some embodiments according to the present invention, a wash solvent used in one of the wash steps may comprise some percentage of acetonitrile, not to exceed 50% v/v.

The sequence of capping and oxidation steps may be reversed, if desired. That is, capping may precede or follow oxidation. Also, with selection of a suitable thiation reagent, the oxidation and capping steps may be combined into a single step. For example, it has been surprisingly found that capping with acetic anhydride may be conducted in the presence of N,N′-dimethyldithiuram disulfide.

Various solvents may be used in the oxidation reaction. Suitable solvents are identified in the Caruthers et al. and Köster et al. patents, cited herein. The Cole et al. patent describes acetonitrile as a solvent for phenylacetyldisulfide. Other suitable solvents include toluene, xanthenes, dichloromethane, etc.

Reagents for cleaving an oligonucleotide from a support are set forth, for example, in the Caruthers et al. and Köster et al. patents, as cited herein. It is considered good practice to cleave oligonucleotide containing thymidine (T) nucleotides in the presence of an alkylated amine, such as triethylamine, when the phosphorus protecting group is O—CH₂CH₂CN, because this is now known to avoid the creation if cyano-ethylated thymidine nucleotides (CNET). Avoidance of CNET adducts is described in general in U.S. Pat. No. 6,465,628, which is incorporated herein by reference, and especially the Examples in columns 20-30, which are specifically incorporated by reference.

The oligonucleotide may be worked up by standard procedures known in the art, for example by size exclusion chromatography, high performance liquid chromatography (e.g. reverse-phase HPLC), differential precipitation, etc. In some embodiments according to the present invention, the oligonucleotide is cleaved from a solid support medium while the 5′-OH protecting group is still on the ultimate nucleoside. This so-called DMT-on (or trityl-on) oligonucleotide is then subjected to chromatography, after which the DMT group is removed by treatment in an organic acid, after which the oligonucleotide is de-salted and further purified to form a final product.

The 5′-hydroxyl protecting groups may be any groups that are selectively removed under suitable conditions. In particular, the 4,4′-dimethoxytriphenylmethyl (DMT) group is a favored group for protecting at the 5′-position, because it is readily cleaved under acidic conditions (e.g. in the presence of dichloroacetic acid (DCA), trichloroacetic acid (TCA), or acetic acid. Removal of DMT from the support-bound oligonucleotide is generally performed with DCA (e.g. about 3 to about 10 percent DCA (v/v) in a suitable solvent. Removal of oligonucleotide after cleavage from the support is generally performed with acetic acid.

As described herein, oligonucleotides can be prepared as chimeras with other oligomeric moieties. In the context of this invention, the term “oligomeric compound” refers to a polymeric structure capable of hybridizing a region of a nucleic acid molecule, and an “oligomeric moiety” a portion of such an oligomeric compound. Oligomeric compounds include oligonucleotides, oligonucleosides, oligonucleotide analogs, modified oligonucleotides and oligonucleotide mimetics. Oligomeric compounds can be linear or circular, and may include branching. They can be single stranded or double stranded, and when double stranded, may include overhangs. In general an oligomeric compound comprises a backbone of linked monomeric subunits where each linked monomeric subunit is directly or indirectly attached to a heterocyclic base moiety. The linkages joining the monomeric subunits, the monomeric subunits and the heterocyclic base moieties can be variable in structure giving rise to a plurality of motifs for the resulting oligomeric compounds including hemimers, gapmers and chimeras. As is known in the art, a nucleoside is a base-sugar combination. The base portion of the nucleoside is normally a heterocyclic base moiety. The two most common classes of such heterocyclic bases are purines and pyrimidines. In the context of this invention, the term “oligonucleoside” refers to nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts.

Synthetic schemes for the synthesis of the substitute internucleoside linkages described above are disclosed in: U.S. Pat. Nos. 5,466,677; 5,034,506; 5,124,047; 5,278,302; 5,321,131; 5,519,126; 4,469,863; 5,455,233; 5,214,134; 5,470,967; 5,434,257. Additional background information relating to internucleoside linkages can be found in: WO 91/08213; WO 90/15065; WO 91/15500; WO 92/20822; WO 92/20823; WO 91/15500; WO 89/12060; EP 216860; PCT/US 92/04294; PCT/US 90/03138; PCT/US 91/06855; PCT/US 92/03385; PCT/US 91/03680; U.S. application Ser. Nos. 07/990,848; 07/892,902; 07/806,710; 07/763,130; 07/690,786; Stirchak, E. P., et al., Nucleic Acid Res., 1989, 17, 6129-6141; Hewitt, J. M., et al., 1992, 11, 1661-1666; Sood, A., et al., J. Am. Chem. Soc., 1990, 112, 9000-9001; Vaseur, J. J. et al., J. Amer. Chem. Soc., 1992, 114, 4006-4007; Musichi, B., et al., J. Org. Chem., 1990, 55, 4231-4233; Reynolds, R. C., et al., J. Org. Chem., 1992, 57, 2983-2985; Mertes, M. P., et al., J. Med. Chem., 1969, 12, 154-157; Mungall, W. S., et al., J. Org. Chem., 1977, 42, 703-706; Stirchak, E. P., et al., J. Org. Chem., 1987, 52, 4202-4206; Coull, J. M., et al., Tet. Lett., 1987, 28, 745; and Wang, H., et al., Tet. Lett., 1991, 32, 7385-7388.

Phosphoramidites used in the synthesis of oligonucleotides are available from a variety of commercial sources (included are: Glen Research, Sterling, Va.; Amersham Pharmacia Biotech Inc., Piscataway, N.J.; Cruachem Inc., Aston, Pa.; Chemgenes Corporation, Waltham, Mass.; Proligo LLC, Boulder, Colo.; PE Biosystems, Foster City Calif.; Beckman Coulter Inc., Fullerton, Calif.). These commercial sources sell high purity phosphoramidites generally having a purity of better than 98%. Those not offering an across the board purity for all amidites sold will in most cases include an assay with each lot purchased giving at least the purity of the particular phosphoramidite purchased. Commercially available phosphoramidites are prepared for the most part for automated DNA synthesis and as such are prepared for immediate use for synthesizing desired sequences of oligonucleotides. Phosphoramidites may be prepared by methods disclosed by e.g. Caruthers et al. (U.S. Pat. No. 4,415,732; 4,458,066; 4,500,707; 4,668,777; 4,973,679; and 5,132,418) and Köster et al. (U.S. RE 34,069).

Double stranded oligonucleotides, such as double-stranded RNA, may be manufactured according to methods according to the present invention, as described herein. In the case of RNA synthesis, it is necessary to protect the 2′-OH of the amidite reagent with a suitable removable protecting groups. Suitable protecting groups for 2′-OH are described in U.S. Pat. Nos. 6,008,400, 6,111,086 and 5,889,136. A particularly suitable 2′-protecting group for RNA synthesis is the ACE protecting group as described in U.S. Pat. No. 6,111,086. In some embodiments, it is considered advantageous to use a different 5′-protecting group for amidites used in RNA synthesis. Suitable 5′-protecting groups are set forth in U.S. Pat. No. 6,008,400. A particularly suitable 5′-protecting group is the trimethylsilyloxy (TMSO) group as taught in U.S. Pat. No. 6,008,400. See especially example 1, columns 10-13. The separate strands of the double stranded RNA may be separately synthesized and then annealed to form the double stranded (duplex) oligonucleotide.

### Oligonucleotide Use

Exemplary preferred antisense compounds include DNA or RNA sequences that comprise at least the 8 consecutive nucleobases from the 5′-terminus of one of the illustrative preferred antisense compounds (the remaining nucleobases being a consecutive stretch of the same DNA or RNA beginning immediately upstream of the 5′-terminus of the antisense compound which is specifically hybridizable to the target nucleic acid and continuing until the DNA or RNA contains about 8 to about 80 nucleobases). Similarly preferred antisense compounds are represented by DNA or RNA sequences that comprise at least the 8 consecutive nucleobases from the 3′-terminus of one of the illustrative preferred antisense compounds (the remaining nucleobases being a consecutive stretch of the same DNA or RNA beginning immediately downstream of the 3′-terminus of the antisense compound which is specifically hybridizable to the target nucleic acid and continuing until the DNA or RNA contains about 8 to about 80 nucleobases). One having skill in the art, once armed with the empirically-derived preferred antisense compounds illustrated herein will be able, without undue experimentation, to identify further preferred antisense compounds.

Antisense and other compounds of the invention, which hybridize to the target and inhibit expression of the target, are identified through experimentation, and representative sequences of these compounds are herein identified as preferred embodiments of the invention. While specific sequences of the antisense compounds are set forth herein, one of skill in the art will recognize that these serve to illustrate and describe particular embodiments within the scope of the present invention. Additional preferred antisense compounds may be identified by one having ordinary skill.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

### RNAse H-Dependent Antisense

One method for inhibiting specific gene expression involves using oligonucleotides or oligonucleotide analogs as “antisense” agents. Antisense technology involves directing oligonucleotides, or analogs thereof, to a specific, target messenger RNA (MRNA) sequence. The interaction of exogenous “antisense” molecules and endogenous mRNA modulates transcription by a variety of pathways. Such pathways include transcription arrest, RNAse H recruitment, and RNAi (e.g. siRNA). Antisense technology permits modulation of specific protein activity in a relatively predictable manner.

### EXAMPLES

The present invention may be further understood with reference to the following, no-limiting, illustrative examples, which may be carried out by methods generally described hereinabove. All references cited herein are expressly incorporated by reference thereto.

### Example 1

### Diethyl Amidite Reagent Synthesis:

1.25 kg (17.09 mol, 2.1 eq.) of diethylamine was mixed with 2 L of hexane and cooled to −78 C. 700 g (4.09 mol, 1 eq.) of the 2-cyanoethyl phosphorodichloridate was added over 30 minutes. The reaction was removed from the cooling bath and stirred for 1 hour. 8 L hexane and 6 L water were added. The aqueous layer was removed and the organic layer was washed 4 times with 5 L 2:3 acetonitrile:water. The organic layer was stripped to give 812 g of 2-cyanoethyl-N,N,N′,N′-tetraethyldiamidite 795 g (3.24 mol, 79% yield).

### General Method of Amidite Syntheses:

The nucleoside was azeotroped 2 times with toluene (1:3 weight to volume) prior to the coupling reaction.

The reaction was done by dissolving the nucleoside in 4 volumes of DMF under Ar and adding the diethyl amidite reagent, 1-H-tetrazole and then N-methyl-imidazole (NMI). The reaction was stirred for 4 hours or until the reaction was complete as determined by TLC (solvent of 15:3:2 EtOAc:DCM:MeOH). 20 mL of TEA was added to the reaction and then transferred to a separatory funnel. The reaction was extracted 3 times with hexane, Toluene with 2% TEA followed by water was added and the lower layer was removed. EtOAC was added and the upper layer was washed with 1:1 DMF:water, 2% TEA, then 9:1 water:brine, 2% TEA, 3 times each. The organic solution was dried over magnesium sulfate, then 20 mL TEA was added and the solution was filtered through a silica pad and stripped. The syrup was precipitated with hexane, re-dissolved with toluene and then re-precipitated with hexane. The final precipitate was dissolved in acetonitrile and stripped to a foam as the final compound.

RNA-G Cpep diethyl amidite
20 g (22 mmol, 1 eq.) nucleoside
8 g diethyl amidite reagent (33 mmol, 1.5 eq.)
0.5 g tetrazole (18 mmol, 0.8 eq.)
0.2 mL N-methyl imidazole (5.6 mmol, 0.25 eq.)
   Final product: 20 g,

RNA-A Cpep diethyl amidite
14.3 g (16 mmol, 1 eq.) nucleoside
5.8 g diethyl amidite reagent (24 mmol, 1.5 eq.)
0.4 g tetrazole (13 mmol, 0.8 eq.)
0.2 mL N-methyl imidazole (5.6 mmol, 0.35 eq.)
   Final product: 10 g

RNA-C Cpep diethyl amidite
50 g (59 mmol, 1 eq.) nucleoside
17 g diethyl amidite reagent (71 mmol, 1.2 eq.)
3.1 g tetrazole (47 mmol, 0.8 eq.)
1.5 mL N-methyl imidazole (19 mmol, 0.25 eq.)
   Final product: 44 g

RNA-U Cpep diethyl amidite
50 g (64 mmol, 1 eq.) nucleoside
19.5 g diethyl amidite reagent (80 mmol, 1.25 eq.)
3.2 g tetrazole (50 mmol, 0.8 eq.)
1.5 mL N-methyl imidazole (19 mmol, 0.25 eq.)
   Final product: 39 g

### EXAMPLE 2

Oligonucleotide synthesis method and conditions:
Synthesizer: ABI 394
Scale: 2 micromoles
Sequence: 5′-U19-moeT-3′
Solid Support: CPG with 2′-O-(2-methoxyethyl)-5-methyl-U (MOE T) loading at 40 micromole/gram
Activator: 0.7 M 2-ethylthiotetrazole in acetonitrile
Detritylation solution: 3% dichloroacetic acid
Cap A solution: 10% acetic anhydride in tetrahydrofuran (THF)
Cap B solution: N-methylimidiazole-pyridine-THF (20:30:50)
Phosphoramidite (10 equivalents)
A: 0.2 M acetonitrile solution of 5′-O-DMT-2′-O-Cpep-3′-O—(B-cyanoethyl-N,N-diethyl)phosphoramidite
B: 0.2 M acetonitrile solution of 5′-O-DMT-2′-O-Cpep-3′-O—(B-cyanoethyl-N,N-diisopropyl)phosphoramidite
C: 0.2 M acetonitrile solution of 5′-O-DMT-2′-O-tBDMS-3′-O—(B-cyanoethyl-N,N-diethyl)phosphoramidite

Three oligonucleotides were synthesized in parallel using phosphoramidites A, B and C under the standard RNA synthetic method. After the solid phase synthesis with Cpep diisopropyl amidite (A) and Cpep diethyl amidite (B), the solid supports was treated with concentrated aqueous ammonia at 55° C. for 15 hours. We found the Cpep diethyl amidite outperformed the Cpep diisopropyl amidite in terms of yield and crude purity (full length oligonucleotide: 80% vs 50%). The diethyl amidite gave the crude 2′-protected oligo (428 O.D.) while the diisopropyl amidite produced 330 O.D. of the crude oligo.

Although certain embodiments have been described through the foregoing Examples, it is to be understood that the present invention is not limited thereto. Indeed the meets and bounds of the present invention are only defined by the following claims, including equivalents.

1-7. (canceled)

8. A compound of the formula: 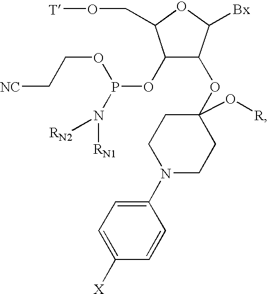
wherein:
T′ is an acid-labile protecting group;
Bx is an optionally protected nucleobase;
R is methyl, ethyl, or n-propyl;
R_{N1} is H, methyl, ethyl, n-propyl or isopropyl;
R_{N2} is, independently of R_{N1} methyl or ethyl;
or together R_{N1} and R_{N2} combine to form a pyrrolidinyl, piperidinyl, morpholino or thiomorpholino group;
and X is an electron-withdrawing group.

9. The compound of claim 8, wherein T′ is 4,4′-dimethoxytriphenylmethyl or pixyl.

10. The compound of claim 8, wherein X is F, Cl, Br or CN.

11. The compound of claim 8, wherein R is ethyl.

12. The compound of claim 8, wherein R_{N1} is methyl, ethyl or isopropyl, and R_{N2} is, independently of R_{N1}, methyl or ethyl.

13. The compound of claim 8, wherein R_{N1} is methyl and R_{N2} is isopropyl.

14. The compound of claim 8, wherein R_{N1} is ethyl and R_{N2} is isopropyl.

15. The compound of claim 8, wherein R_{N1} and R_{N2} together form a pyrrolidinyl or morpholino moiety.

16. The compound of claim 8, wherein Bx is T, U or optionally protected G, A, C or 5-methyl C.

17. The compound of claim 8, wherein Bx is T.

18. The compound of claim 8, wherein Bx is U.

19. The compound of claim 8, wherein Bx is optionally protected G.

20. The compound of claim 8, wherein Bx is optionally protected A.

21. The compound of claim 8, wherein Bx is optionally protected C or 5-methyl C.

22. The compound of claim 8, wherein Bx is protected G.

23. The compound of claim 22, wherein Bx is G protected with phenylacetyl.

24. The compound of claim 8, wherein Bx is protected A.

25. The compound of claim 24, wherein Bx is A protected with pivolyl.

26. The compound of claim 8, wherein Bx is protected C or protected 5-methyl C.

27. The compound of claim 25, wherein Bx is C or 5-methyl C protected with phenylacetyl.

28-38. (canceled)

39. A process comprising:
providing a support-bound species of the formula: 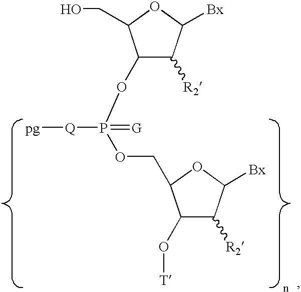
wherein:
n is 0 or a positive integer from 1 to 100;
each Bx is an optionally protected nucleobase;
each G is O or S;
each Q is O or S;
each pg is H or a protecting group;
each R_{2′} is H, a 2′-deoxy-2′-substituent, or a protected OH group; and
T′ is a support medium or a linker covalently linked to a support medium;
reacting said support-bound species with an amidite of formula: 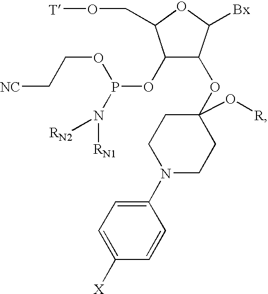
wherein:
T′ is an acid-labile protecting group;
Bx is an optionally protected nucleobase;
R is methyl, ethyl, or n-propyl;
R_{N1} is H, methyl, ethyl, n-propyl or isopropyl;
R_{N2} is, independently of R_{N1} methyl or ethyl;
or together R_{N1} and R_{N2} combine to form a pyrrolidinyl, piperidinyl, morpholino or thiomorpholino group; and
X is an electron-withdrawing group;
to form a support-bound phosphityl compound of formula: 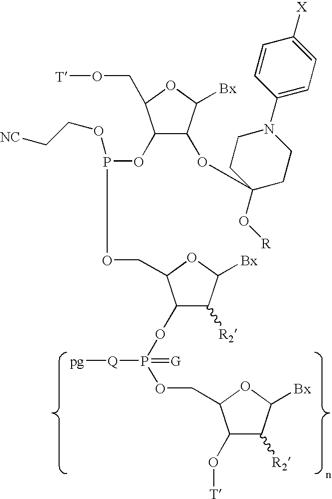
and
oxidizing or sulfurizing the support-bound phosphityl compound to form a phosphotriester compound of formula:

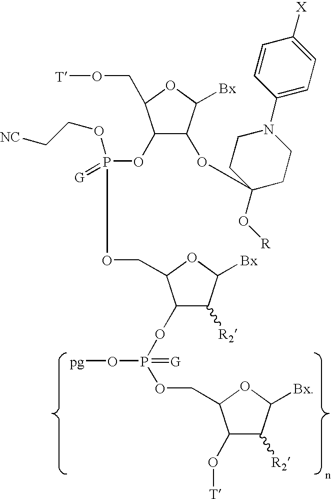

40. The process of claim 39, wherein R is ethyl.

41. The process of claim 39, wherein Bx is U, T or optionally protected G, A, C or 5-methyl C.

42. The process of claim 39, wherein Bx is optionally protected G.

43. The process of claim 39, wherein Bx is optionally protected A.

44. The process of claim 39, wherein Bx is optionally protected C or 5-methyl C.

45. The process of claim 39, wherein Bx is U or T.

46. The process of claim 39, wherein R_{N1} is methyl, ethyl or isopropyl, and R_{N2} is, independently of R_{N1}, methyl or ethyl.

47. The process of claim 39, wherein R_{N1} is methyl and R_{N2} is isopropyl.

48. The process of claim 39, wherein R_{N1} is ethyl and R_{N2} is isopropyl.

49. The process of claim 39 wherein each Q is O, and each pg is cyanoethyl.

50. The process of claim 39 further comprising repeating steps (a)-(c) a plurality of times.

51. The process of claim 39 further comprising cleaving the phosphotriester compound from the support medium.

52. The process of claim 39 further comprising the step of (d) capping unreacted support bound hydroxyl groups.
